(19) United States
(12) Patent Application Publication
(10) Pub. No.: US 2026/0181073 A1
(43) Pub. Date: Jun. 25, 2026

(54) EFFICIENT DATA SHARING ACROSS DISPARATE SYSTEMS

(71) Applicant: MOTOROLA SOLUTIONS, INC., Chicago, IL (US)

(72) Inventors: Srividya G. SUBRAMANYAM, South Barrington, IL (US); Taylor CASTON, Marietta, GA (US); Noemi ANDRAS, Morton Grove, IL (US)

(21) Appl. No.: 18/999,042

(22) Filed: Dec. 23, 2024

(51) Int. Cl. H04M 3/42; G16H 10/60; G16H 40/20; G16H 80/00; H04M 11/04

(52) U.S. Cl. CPC H04M 3/42068; G16H 10/60; G16H 40/20; G16H 80/00; H04M 11/04

(57) ABSTRACT

A method for sharing data across disparate emergency provider systems is described. The method comprising: analyzing content of communications between a caller and a call taker. An identifier associated with the caller is determined. An escrow database is accessed to determine the presence of a behavioural assessment indicator (BAI). The BAI indicates that the caller has a mental health profile stored in the escrow database. Permission is received from the caller to access the mental health profile. Mental health information of the caller is retrieved from a data structure comprising a first portion storing a locked mental health history and a second portion storing accessible mental health status information. The retrieved information is presented to the call taker to assist handling the call. A device comprising a processor and a computer-readable storage medium storing instructions that cause the processor to perform the method is also described.

### BACKGROUND

Emergency crisis prevention call centers, such as the 988 Suicide and Crisis Lifeline, offer callers access to trained crisis counselors who can help people experiencing suicidal thoughts, substance use, mental health crises, or any other kind of emotional distress.

Emergency dispatch centers, such as the 911 Universal Emergency Number, offer callers direct access to call takers who are the first point of contact during an emergency situation. The call takers answer calls, assess the situation, and determine the appropriate response. They also provide instructions to the caller on what to do. Call takers use computers to record information and route calls to a dispatcher. The dispatchers dispatch the relevant law enforcement, fire prevention, or medical agency to the caller.

However, the emergency crisis prevention call centers and emergency dispatch centers are managed differently. Further, user data associated with the callers is not shared between the centers. Accordingly, the dispatchers typically have no insight into the caller's mental health history. Thus, the inability to efficiently share data across disparate systems may put a caller's life at risk.

### BRIEF DESCRIPTION OF THE SEVERAL VIEWS OF THE DRAWINGS

In the accompanying figures similar or the same reference numerals may be repeated to indicate corresponding or analogous elements. These figures, together with the detailed description, below are incorporated in and form part of the specification and serve to further illustrate various embodiments of concepts that include the claimed invention, and to explain various principles and advantages of those embodiments.

FIG. 1 is a block diagram of an example system for efficiently sharing data across disparate emergency provider systems.

FIG. 2 is a block diagram of a crisis prevention center, in accordance with some examples.

FIG. 3 is a block diagram of an emergency dispatch center, in accordance with some examples.

FIG. 4 is a block diagram showing a device structure of a call center computer, in accordance with some examples.

FIG. 5a is a flowchart illustrating a general method for processing a call at the crisis prevention center, in accordance with some examples.

FIG. 5b is a flowchart illustrating a general method for processing a call at the emergency dispatch center, in accordance with some examples.

FIG. 6 is a flowchart illustrating a general method for accessing the escrow database, in accordance with some examples.

FIG. 7 is a flowchart illustrating a general method for confirming information submitted to the escrow database, in accordance with some examples.

FIG. 8 is an example of a graphical representation of mental health information.

Skilled artisans will appreciate that elements in the figures are illustrated for simplicity and clarity and have not necessarily been drawn to scale. For example, the dimensions of some of the elements in the figures may be exaggerated relative to other elements to help improve understanding of embodiments of the present disclosure.

The system, apparatus, and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION OF THE INVENTION

A “988” call in the United States refers to a call to the Suicide and Crisis Lifeline, which is a free, and confidential support system for individuals experiencing mental health crises, suicidal thoughts, or emotional distress. The Suicide and Crisis Lifeline provides crisis support to callers. For example, it provides direct assistance for individuals in suicidal crises or emotional distress. It also provides mental health resources. Callers are connected to trained counselors who offer support and suggest resources or interventions. Finally, emergency intervention may be provided, if necessary, in which counselors dispatch crisis teams or other emergency services to ensure the safety of the caller.

Emergency dispatch centres, such as a “911” call in the United States, provides response for immediate medical emergencies, crimes in progress, or life-threatening situations. Emergency responders like police or EMS may not have specialized mental health training, which is why 988 was created to better address mental health needs.

Since the purpose of each of the systems is different, they are managed separately. The closest the systems have come to integration is by providing referrals. For example, if a 988 caller's situation escalates to an immediate physical threat to themselves or others, the counselors may transfer the call to 911. This usually involves sharing critical information about the caller, such as location and the nature of the emergency, to ensure first responders can assist effectively. However, historical information about the caller is not available to be shared.

Similarly, in non-life-threatening situations involving mental health, 911 call takers may transfer the caller to the Suicide and Crisis Lifeline to connect them with mental health professionals. Again, the decision must be made by the call taker without any historical information about the caller.

In accordance with an aspect of the present specification, there is provided a method comprising: analyzing, at a first emergency services provider, content of communications between a caller experiencing an event and call taker; determining an identifier associated with the caller; accessing an escrow database using the identifier associated with the caller to determine the presence of a behavioural assessment indicator, the behavioural assessment indicator indicating that the caller has a mental health profile stored in the escrow database, wherein the escrow database is generally inaccessible but becomes accessible by predefined, disparate service providers responsive to the caller contacting the first emergency services provider; receiving permission from the caller to access the mental health profile; retrieving mental health information of the caller from a data structure in the mental health profile, the data structure comprising a first portion storing a locked mental health history of the caller and a second portion storing accessible information including the information about a mental health status of the caller; presenting, to the call taker, the information about the mental health status of the caller to assist the call taker in handling the call.

In accordance with another aspect of the present specification, there is provided a device comprising a processor and a computer readable medium having stored thereon program instructions that, when executed by the processor, cause the processor to perform a set of operations as described above.

In some embodiments, the information from the mental health profile may be presented to the call taker dynamically with progression of the communication based on the analysis of the content thereof.

In some embodiments, the content of communications may be analyzed to identify additional mental health information about the mental health status of the caller. The escrow database may be accessed to update the mental health profile of the caller with the additional mental health information. An indication may be received that the caller has not requested anonymity. In response to the indication, the communication may be analyzed to identify additional information for including in the mental health history. The escrow database may be accessed to update the first portion of the metal health profile of the caller with the additional information.

In some embodiments, the call taker may be presented with at least one mental health code based on the analysis of the communication. The call taker may also be presented with a plurality of tags associated with at least one mental health code. The tags provide a simplified, readable representation of a mental health condition of the caller. The tags may include predefined tags and customized tags.

In some embodiments, an indication that the additional mental health information was identified may be sent to the communication device of the caller. The indication includes an actionable link allowing the caller to review at least a portion of the additional mental health information. Upon approval by the caller, the additional mental health information may be stored in the escrow database.

In some embodiments, presenting the mental health profile includes presenting mental health codes, tags, and call history stored in the mental health profile. The tags provide a simplified, readable representation of a mental health condition of the caller. The mental health codes may be specific to a region from which the mental health codes were originally submitted to the escrow database. The mental health codes may be converted to local mental health codes specific to a region in which the call taker is located. The local mental health codes may be presented to the call taker.

In some embodiments, an indication may be received that the caller has not requested anonymity. In response to the indication, the mental health history of the caller may be retrieved from the data structure in the mental health profile. The mental health history of the caller may be presented to the call taker.

In some embodiments, an actionable mental health care assist button may be presented to the call taker. When actioned, the button causes a mental health care professional associated with the stored mental health codes to be conferenced into call.

In some embodiments, a graphical representation of the mental health codes may be generated for presenting to the call taker in addition to the mental health codes and the tags associated with the mental health codes. If the indication that the caller has not requested anonymity is received, the mental health history of the caller may be included in the dynamically generated graphical representation. The mental health codes and the tags associated with the mental health codes are displayed so that hovering over them provides an expanded description thereof to the call taker.

Example embodiments are herein described with reference to flowchart illustrations and/or block diagrams of methods, apparatus (systems) and computer program products according to example embodiments. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a special purpose and unique machine, such that the instructions, which execute via the processor of the computer or other programmable data processing apparatus, create means for implementing the functions/acts specified in the flowchart and/or block diagram block or blocks. The methods and processes set forth herein need not, in some embodiments, be performed in the exact sequence as shown and likewise various blocks may be performed in parallel rather than in sequence. Accordingly, the elements of methods and processes are referred to herein as “blocks” rather than “steps.”

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the flowchart and/or block diagram block or blocks.

The computer program instructions may also be loaded onto a computer or other programmable data processing apparatus that may be on or off-premises, or may be accessed via the cloud in any of a software as a service (SaaS), platform as a service (PaaS), or infrastructure as a service (IaaS) architecture so as to cause a series of operational blocks to be performed on the computer or other programmable apparatus to produce a computer implemented process such that the instructions which execute on the computer or other programmable apparatus provide blocks for implementing the functions/acts specified in the flowchart and/or block diagram block or blocks. It is contemplated that any part of any aspect or embodiment discussed in this specification can be implemented or combined with any part of any other aspect or embodiment discussed in this specification.

Further advantages and features consistent with this disclosure will be set forth in the following detailed description, with reference to the figures.

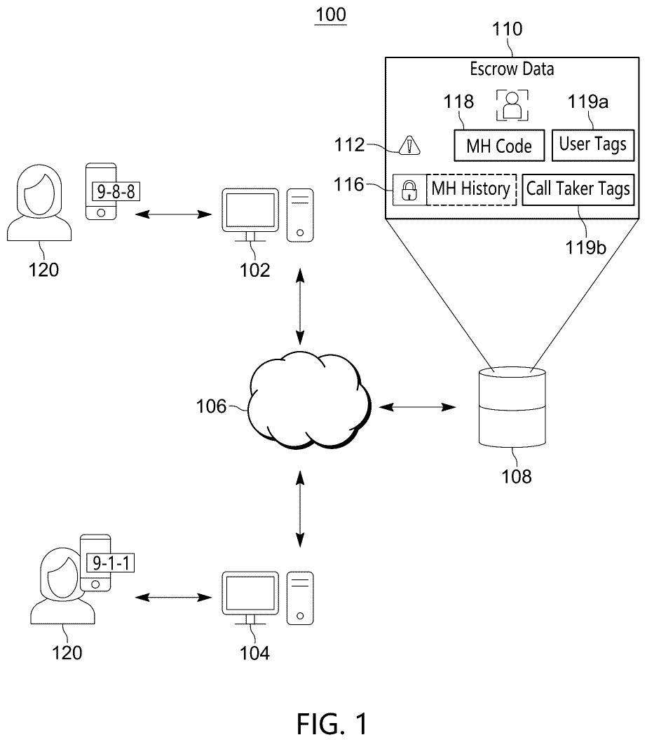

Referring to FIG. 1, an example system for efficiently sharing data across disparate emergency provider systems is illustrated generally by numeral 100. The system 100 includes a crisis prevention call center 102, an emergency dispatch center 104, a communication network 106, and an escrow database 108. The escrow database 108 includes a plurality of user profiles 110. Each user profile 110 stores a behavioral assessment indicator (BAI) 112. Each user profile 110 may also include a metal health profile of the user. The mental health profile stores, in a data structure, historical mental health information about the caller. The data structure includes a first portion storing locked information and a second portion storing accessible information. The locked information includes personal information about the caller. In some embodiments, the locked information includes some or all of a mental health history 116 of the caller. The mental heath history refers to a personal record of the caller's past and present experiences, diagnoses, symptoms, treatments, and outcomes related to their mental health. The accessible information about the caller may include general information about a mental health status of the caller. In some embodiments, the information includes mental health codes 118 and readable tags associated with the user profile. The mental health codes 118 are alphanumeric codes used in medical billing, documentation, and research to classify mental health conditions and services. The tags may include user tags 119a and call taker tags 119b. The tags provide a simplified, readable representation of a mental health condition of the caller 120. In some embodiments, the accessible information may also include a call history. The call history may include a list of calls that the caller 120 has made to crisis prevention centers 102 and/or emergency dispatch centers 104. Accordingly, it will be appreciated that while the mental health history 116 is personalized and specific to the caller's experiences, the mental health codes 118 serve as a standardized framework for describing and managing mental health information within the healthcare system.

The crisis prevention call center 102 receives calls from callers 120 experiencing mental health issues. The emergency dispatch center 104 receives calls from callers 120 that require immediate emergency services.

The escrow database 108 securely holds and manages data on behalf of the crisis prevention call center 102 and the emergency dispatch center 104. This arrangement ensures that critical information is safely stored and can be accessed or released only under specific, predefined conditions. Accordingly, the escrow database 108 is generally inaccessible (to anyone except perhaps the caller) but may become accessible in response to the caller 120 contacting one of the crisis prevention call center 102 or the emergency dispatch center 104. For example, in some embodiments the escrow database 108 may be stored on the caller's device. The caller's device forwards information from the data structure in the mental health profile to the call taker's computing device. The information may be forwarded responsive to 911/988 being dialed or otherwise contacted. In some embodiments the escrow database 108 may be stored on a cloud-based third-party system. For example, the escrow database 108 may be integrated into a Smart911™ database. The caller's device or the call taker's device may trigger the cloud-based system store to forward the data structure in the mental health profile to the call taker. Alternatively, the caller's device or call taker's device may actively seek out and retrieve the data structure from the mental health profile in the third-party system. Other possibilities for storing and accessing the data structure in the mental health profile are also possible.

The BAI 112 indicates whether the user profile 110 associated with the caller 120 has a mental health profile stored in the data escrow database 108. In some embodiments, the BAI 112 is a flag. If the flag is set, there is a mental health profile. If the flag is not set, there is no mental health profile. The BAI 112 allows the crisis prevention call center 102 and the emergency dispatch center 104 to readily determine whether the mental health profile 110 is available for the caller 120.

The data structure addresses concerns about privacy of the mental health profile 110. Specifically, the data structure includes a first portion storing locked information including the mental health history 116 of the caller 120, which is lockable by the caller 120. In some embodiments, the mental health history 116 requires further authorization from the caller 120 before access to the information stored therein is permitted. The data structure includes a second portion storing accessible information about a mental health status of the caller 120.

The communication network 106 facilitates communication between the crisis prevention center 102, the emergency dispatch center 104, and the escrow database 108. In some embodiments, the communication network 106 includes wired and wireless broadband technologies to facilitate communication over wide area networks (WANs), wireless wide area networks (WWANs), local area networks (LANs) and/or wireless local area networks (WLANs). WWANs use mobile communication technologies like 3G, 4G LTE, 5G, 6G and the like to connect devices to a network, such as the Internet. WLANs use radio waves to connect the devices to the network. At present this is typically done using the IEEE 802.11 Wi-Fi standards.

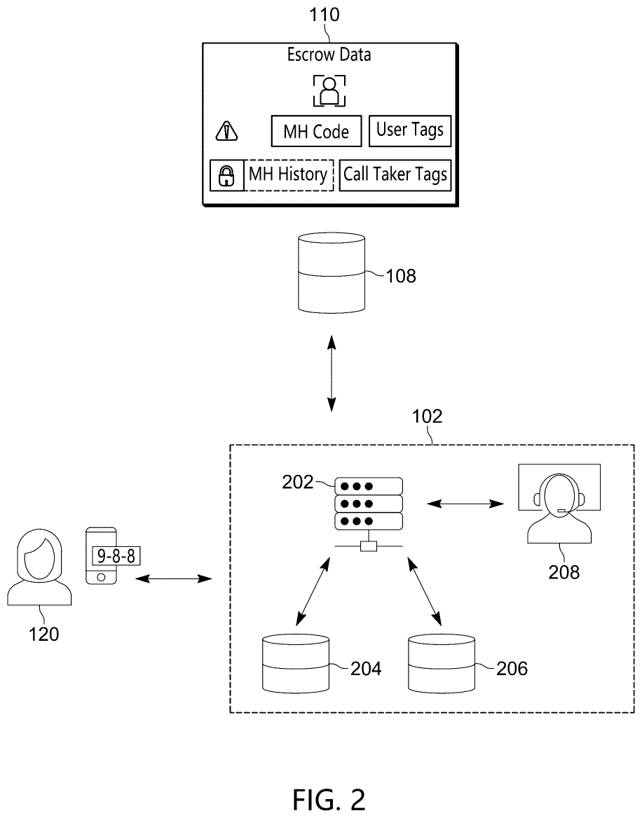

Referring to FIG. 2, the crisis prevention center 102 is illustrated in greater detail. The crisis prevention center 102 includes a call center computer 202, a crisis prevention case database 204, and a mental health resource database 206. A crisis prevention call taker 208 is communicatively coupled with the call center computer 202, which provides the call taker 208 with access to the crisis prevention case database 204, the mental health resource database 206, as well as the escrow server 108.

The case database 204 includes case information about the caller 120, including the caller's phone number, case status, and any case notes. The case database 204 is only available to the crisis prevention center 102 and not to the emergency dispatch center 104. The mental health resource database 206 includes a list of mental health codes and their associated definitions. In some embodiments, the mental health resource database 206 is part of a third-party system, such as computer aided dispatch (CAD) system, which may also be accessible by the emergency dispatch center 104.

In some embodiments, the mental health codes are defined regionally. Accordingly, the mental health resource database 206 includes a plurality of different sets of mental health codes. The set of mental health codes that is made available to the call taker 208 depends on the location of the caller 120 and/or the call taker 208. In some embodiments, the mental health resource database 206 includes a mapping that correlates the mental health codes in different regions. The mapping can be used to translate between the mental health codes in different regions, as will be described.

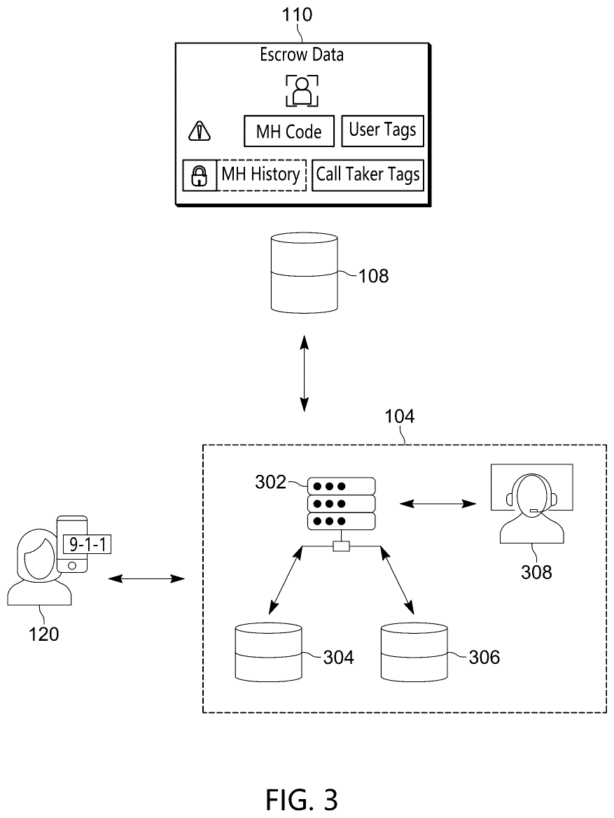

Referring to FIG. 3, the emergency dispatch center 102 is illustrated in greater detail. The emergency dispatch center 104 includes a call center computer 302, an emergency dispatch case database 304, and an emergency dispatch resource database 306. An emergency dispatch call taker 308 is communicatively coupled with the call center computer 302, which provides the call taker 308 with access to the emergency dispatch case database 304, the emergency dispatch resource database 306, as well as the escrow server 108.

The case database 304 includes case information about the caller 120, including the caller's phone number, case status, and any case notes. The case database 304 is only available to the emergency dispatch center 104 and not to the crisis prevention center 102. The emergency dispatch resource database 306 includes a list of mental health codes and their associated definitions. In some embodiments, the emergency dispatch resource database 306 is part of a third-party system, such as CAD system, which may also be accessible by the crisis prevention center 102. The CAD system can also provide the emergency dispatch center 104 with other resources, such as first responder status, mental health assist status, and call divert status. The emergency dispatch database 306 may also include the mapping that correlates the mental health codes in different regions.

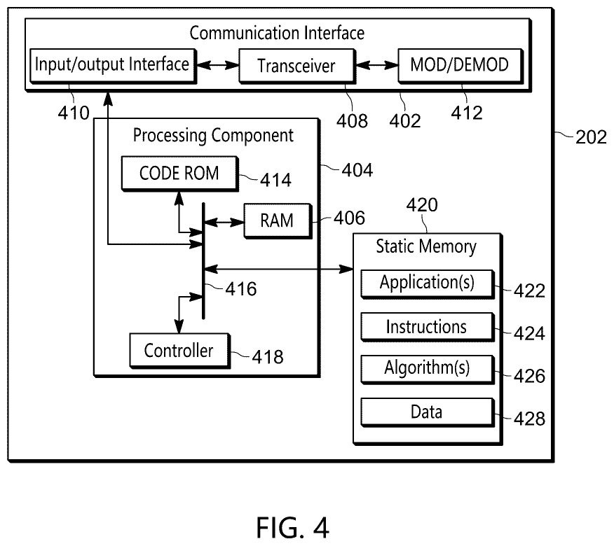

Referring to FIG. 4, a schematic block diagram of an example of the call center computer 202 is illustrated. The call center computer 202 may include a communication interface 402, a processing component 404, and a memory 420.

The communication interface 402 includes one or more transceivers 408, input/output (I/O) interfaces 410, and modems 412. The I/O interfaces 410 may include one or more wired and/or wireless interfaces that are configurable to communicate with suitable components of the call center computer 202. For example, the I/O interfaces 410 may interface with a display, keypad, camera, and/or microphone with the call taker 208, 308.

The one or more transceivers 408 may be adapted for communication with the communication network 106 for communicating with the callers 120, the escrow database 108, and/or with other call center computers 202. For example, the one or more transceivers 408 may be adapted for communication with one or more of the Internet, a Bluetooth network, a Wi-Fi network, for example operating in accordance with the IEEE 802.11 standards, an LTE (Long-Term Evolution) network and/or other types of GSM (Global System for Mobile communications) and/or 3GPP (3rd Generation Partnership Project) networks, a 5G network (e.g., a network architecture compliant with, for example, the 3GPP TS 23 specification series and/or a new radio (NR) air interface compliant with the 3GPP TS 38 specification series) standard, a Worldwide Interoperability for Microwave Access (WiMAX) network, for example operating in accordance with an IEEE 802.16 standard, and/or another similar type of wireless network.

Hence, the one or more transceivers 408 may include a cell phone transceiver, a 3GPP transceiver, an LTE transceiver, a GSM transceiver, a 5G transceiver, a Bluetooth transceiver, a Wi-Fi transceiver, a WiMAX transceiver, and/or another similar type of wireless transceiver configurable to communicate via a wireless radio network.

The communication interface 402 may further include one or more wireline transceivers 408, such as an Ethernet transceiver, a USB (Universal Serial Bus) transceiver, or similar transceiver configurable to communicate via a twisted pair wire, a coaxial cable, a fiber-optic link, or a similar physical connection to a wireline network. The transceiver 408 may also be coupled to the modem 412.

The processing component 404 may include a Read Only Memory (ROM) 414, a Random Access Memory (RAM) 406, a common data and address bus 416 and a controller 418. The ROM 414, the RAM 406, and the controller 418 are communicatively coupled to the common data and address bus 416. Further, the communication interface 402 and the memory 420 are communicatively coupled to the processing component 404 via the common data and address bus 416.

The controller 418 may include one or more logic circuits, one or more processors, one or more microprocessors, one or more GPUs (Graphics Processing Units), and/or the controller 418 may include one or more ASICs (application-specific integrated circuits), one or more FPGA (field-programmable gate arrays), and/or another electronic device.

The memory 420 provides a non-transitory computer readable medium for storing machine readable instructions to implement one or more programs or applications 422, instructions or program code 424, algorithms 426, and/or application data 428. While the instruction or program code, 424, algorithms 426, and the application data 428 are shown as being separate from the applications 422, they may be components of the applications 422.

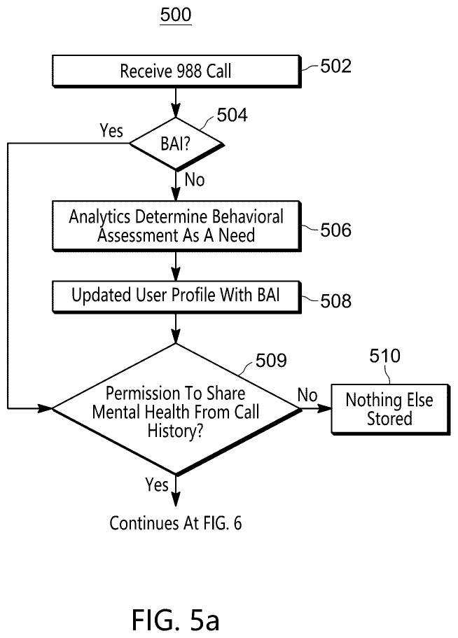

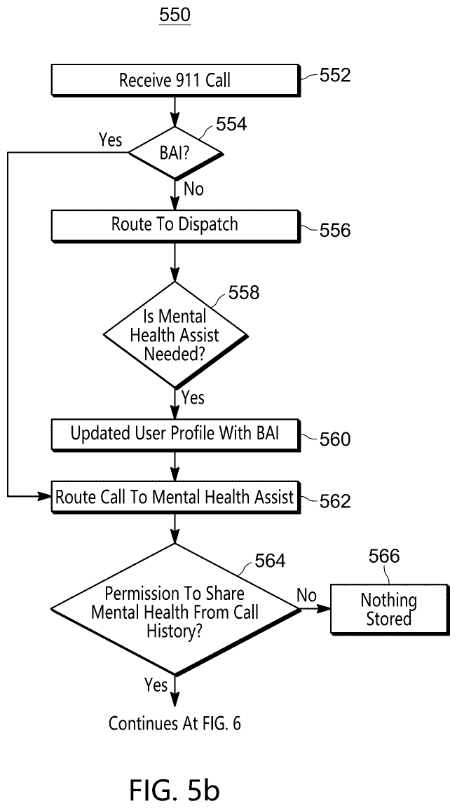

Thus, for example, the memory 420 stores instructions and/or program code 424 and/or algorithms 426 and/or data 428 corresponding to the at least one application 422 that, when executed by the controller 418, enables the controller 418 to implement functionality for efficiently sharing data across disparate systems, including but not limited to, one or more or all of the blocks of the method set forth in FIG. 5a to FIG. 7.

Put another way, the memory 420 may comprise a non-transitory computer-readable storage medium having stored thereon program instructions that, when executed by the controller 418, cause the controller 418 to perform a set of operations comprising one or more or all of the blocks of the method set forth in FIG. 5a to FIG. 7.

The application 422 and/or one or more of the algorithms 426 may include programmatic algorithms, and the like, to implement functionality as described herein.

Alternatively, and/or in addition to programmatic algorithms, the application and/or the one or more of the algorithms may include one or more machine learning algorithms to implement functionality as described herein.

The one or more machine learning algorithms of the application and/or the algorithms may include, but are not limited to: a deep-learning based algorithm; a neural network; a generalized linear regression algorithm; a random forest algorithm; a support vector machine algorithm; a gradient boosting regression algorithm; a decision tree algorithm; a generalized additive model; evolutionary programming algorithms; Bayesian inference algorithms, reinforcement learning algorithms, and the like. Any suitable machine learning algorithm and/or deep learning algorithm and/or neural network is within the scope of present examples.

Furthermore, in examples where the application and/or the one or more of the algorithms includes one or more machine learning algorithms, the application and/or the one or more of the algorithms may be operated in a training mode to train the application and/or the one or more of the algorithms to implement the functionality described herein.

Referring to FIG. 5a to FIG. 7, flowcharts representative of methods for sharing data across disparate emergency provider systems are illustrated. The operation of the methods corresponds to machine readable instructions that are executed by the controller 418. In the illustrated examples, the instructions represented by the blocks are stored at the memory 420 for example, as the application 422 and/or the algorithms 426. The methods are some ways in which the controller 418 may be configured.

The methods need not be performed in the exact sequence as shown and likewise various blocks may be performed in parallel rather than in sequence. Accordingly, the elements of the methods are referred to herein as “blocks” rather than “steps”. The methods may be implemented on variations of the system 100 of FIG. 1, as well.

Furthermore, in the description of the methods, reference is made to at least one computing device performing the method as functionality indicated by the method may be performed in a distributed manner.

Referring to FIG. 5a, a flowchart representative of a general method for processing a call at the crisis prevention center 102 is illustrated generally by numeral 500. At block 502 a call is received at the crisis prevention center 102 from the caller 120. At block 504, it is determined whether the caller 120 has a user profile 110 with a BAI indicator 112. The call center computer 202 accesses the user profile 110 of the caller 120 at the escrow database 108. Although the escrow database 108 is generally inaccessible, it is accessible to the crisis prevention center 102 responsive to the call from the caller 120. The caller 120 is identified by an associated identifier. In some embodiments, the identifier is a telephone number of the caller 120 or a hardware or network MAC address assigned to the caller's mobile phone. In some embodiments, the identifier is a unique user identifier assigned to the caller 120. The user identifier is used to identify the caller 120 when the caller 120 contacts the crisis prevention center 102 via an app on a smart phone rather than using a traditional telephone connection, for example.

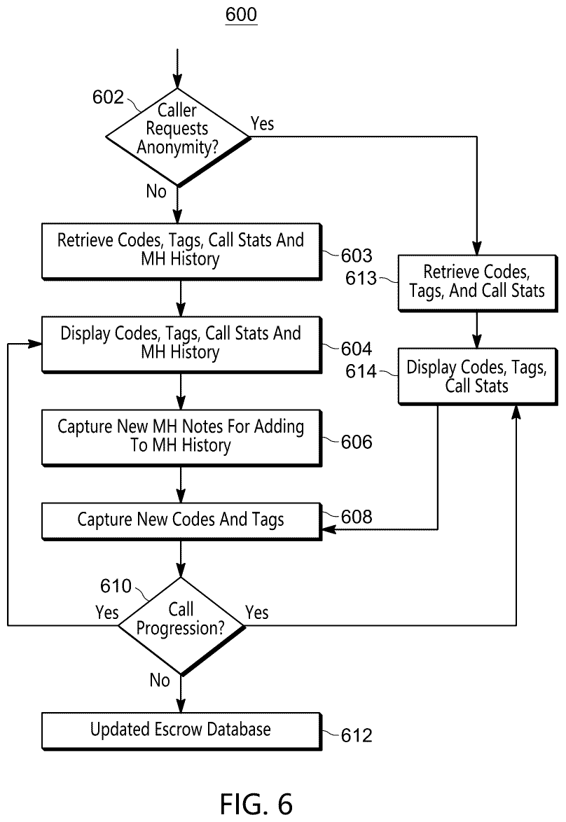

If the BAI 112 indicates that a mental health profile is not present, then at block 506, call analytics analyse the content of the communication (e.g., via natural language processing or text processing after a voice to text conversion) until it determines that a behavioural assessment is necessary. At block 508, the user profile 110 of the caller 120 is updated so that the BAI 112 is set. At block 509, permission is requested from the caller 120 to store mental health information associated with the caller into the escrow database 108. In some embodiments, permission may be requested from the caller 120 during the call. In some embodiments, permission may be granted by the caller 120 prior to the call. The permission may be stored in the user profile 110 in the escrow database 108. If permission is not granted then at block 510, the call continues. However, nothing from the communication between the caller 120 and call taker 208, other than the BAI 112, is stored to the escrow database 108. If permission is granted, then the method continues as described in FIG. 6.

Returning to block 504, if the BAI indicates that a mental health profile is present, at block 508, permission is requested from the caller 120. Since a mental health profile is present, permission is requested to retrieve mental health information from the escrow database 108 as well as to store the mental health information into the escrow database 108.

Referring to FIG. 5b, a flowchart representative of a general method for processing a call at the emergency dispatch center 104 is illustrated generally by numeral 550. At block 552 a call is received at the emergency dispatch center 104 from the caller 120. At block 554, it is determined whether the caller 120 has a user profile 110 with a BAI indicator 112. The call center computer 202 accesses the user profile 110 of the caller at the escrow database 108. Although the escrow database 108 is generally inaccessible, it is accessible to the emergency dispatch center 102 responsive to the call from the caller 120. The caller 120 is identified by an associate identifier. In some embodiments, the identifier is a telephone number of the caller 120 or a hardware or network MAC address assigned to the caller's mobile phone. In some embodiments, the identifier is a unique user identifier assigned to the caller 120. The user identifier is used to identify the caller 120 when the caller 120 contacts the emergency dispatch center 104 via an app on a smart phone rather than using a traditional telephone connection, for example.

If the BAI 112 indicates that a mental health profile is not present, then at block 556, the call is routed to dispatch. At block 558, during the course of the call, if the dispatcher determines that a mental health assist is needed (e.g., via natural language processing or text processing after a voice to text conversion) then at block 560 the user profile 110 of the caller 120 is updated so that the BAI 112 is set. At block 562, the call is routed to mental care assist. At block 564, permission is requested from the caller 120 to store mental health information associated with the caller into the escrow database 108. In some embodiments, permission may be requested from the caller 120 during the call. In some embodiments, permission may be granted by the caller 120 prior to the call. The permission may be stored in the user profile 110 in the escrow database 108. If permission is not granted then at block 566, the call continues. However, nothing from the communication between the caller 120 and call taker 308 is stored to the escrow database 108. If permission is granted, then the method continues as described in FIG. 6.

Returning to block 554, if the BAI indicates that a mental health profile is present, then at block 562, the call is routed to mental care assist. At block 564 permission is requested from the caller 120. Since a mental health profile is present, permission is requested to retrieve mental health information from the escrow database 108 as well as to store the mental health information into the escrow database 108.

Referring to FIG. 6, a flowchart representative of a general method for accessing the escrow database 108 is illustrated generally by numeral 600. The method may be implemented by the crisis prevention center 102 and/or the emergency dispatch center 104, referred to generically as an emergency services provider. As a reminder, before implementing the general method 600 for accessing the escrow database 108, it has already been determined, either at block 509 or block 564 that permission has been granted by the caller for the call taker to store mental health information associated with the caller into or retrieve mental health information associated with the caller from the escrow database 108. At block 602, it is determined whether the caller 120 wishes to request anonymity. In some embodiments, the request for anonymity may be made by the caller 120 during the call. In some embodiments, the request for anonymity may be made the caller 120 prior to the call. The request for anonymity may be stored in the user profile 110 in the escrow database 108.

If the caller requests anonymity, then at block 613 information about a mental health status of the caller is retrieved from the data structure in the mental health profile 110. Specifically, the information is retrieved from the accessible information stored in the second portion of the data structure. In some embodiments, the accessible information includes a list of the mental health codes 118, the user tags 119a, the call taker tags 119b, and the call history associated with the caller 120. Note that the mental health history 116 from the first portion of the data structure is not retrieved since the caller has requested anonymity.

At block 614, a graphical representation of the mental health codes 118, the user tags 119a, the call taker tags 119b, and the call history is presented to the call taker 208, 308 to assist the call taker 208, 308 in handling the call. The graphical representation includes the mental health codes 118, the user tags 119a, the call taker tags 119b, the call history, as well as a visual summary of the information. The mental health codes 118 and the tags 119a, 119b are displayed so that hovering over them provides an expanded description thereof to the call taker 208, 308.

In some embodiments, the graphical representation further includes an actionable mental health care assist button. When actioned, the mental health care assist button causes a mental health care professional associated with one or more of the retrieved mental health codes 118 to be conferenced into call.

As previously described, the mental health codes 118 may be specific to the region from which the mental health codes 118 were originally submitted to the escrow database 108. For convenience this region will be referred to as the originating region.

In some embodiments, the originating region of the mental health codes 118, the user tags 119a, and the call taker tags 119b is also displayed to the call taker. In some embodiments, if it is determined that the call taker is outside of the originating region, the mapping that correlates the mental health codes 118 in different regions is retrieved. The mapping is used to convert the mental health codes 118 to local mental health codes specific to a region in which the call taker is located. The local health codes 118 can then be presented to the call taker.

In some embodiments, only relevant information from the mental health profile is presented to the call taker based on the relevance to the conversation between the caller and the call taker. For example, the conversation between the caller and the call taker can be analyzed via natural language processing or text processing after a voice to text conversion. It may be determined that the call taker has asked the caller a specific question. For example, if it is determined that the caller 120 is feeling anxious or if it is determined that the conversation between the caller and the call taker related to anxiety, then the health codes 118, user tags 119a, and call taker tags 119b from the data structure that are relevant to anxiety are presented to the call taker.

At block 608, the content of the call (for example via natural language processing or text processing after a voice to text conversion) is further analyzed to identify additional information about the mental health status of the caller. In some embodiments, the content of the call may include questions asked by the call taker and the caller's corresponding responses. The call taker is presented with at least one mental health code 118 based on the analysis of the call. The call taker selects one of more of the mental health codes 118 that apply to the caller. In some embodiments, the call taker is also provided with a plurality of call taker tags 119b associated with the mental health codes 118. The call taker tags 119b provide a simplified, readable representation of the mental health condition of the caller. The call taker selects one or more of the call taker tags 119b that apply to the caller. In some embodiments, the call taker can enter a customized call taker tag 119b, if none of the predefined call taker tags 119b accurately represent the mental health condition of the caller.

At block 610 it is determined if the call has progressed (via natural language processing or text processing after a voice to text conversion). For example, the call taker may have asked the caller 120 another question. If the call has progressed, the method returns to block 614 since the caller has requested anonymity. The mental health codes 118, the user tags 119a, the call taker tags 119b, and the call history presented to the call taker are updated to address the progression of the call. Thus, the presentation of the information from the mental health profile of the caller is displayed to the call taker dynamically with progression of the call. In this way, the most relevant information about the mental health status of the caller 120 is presented to the call taker at the appropriate time.

Returning to block 602, if the caller does not request anonymity, then at block 603 the mental health information about the caller is retrieved from the data structure. As in block 613, information about the mental health status of the caller is retrieved from the data structure in the mental health profile 110. Specifically, the information is retrieved from the accessible information stored in the second portion of the data structure. In some embodiments, the accessible information includes a list of the mental health codes 118, the user tags 119a, the call taker tags 119b, and the call history associated with the caller 120. In addition, since the caller has not requested anonymity, in this instance the mental health history 116 is also retrieved. Specifically, the mental health history is unlocked and retrieved from the first portion of the data structure.

At block 604, as in block 614, the graphical representation of the mental health codes 118, the user tags 119a, the call taker tags 119b, and the call history is presented to the call taker 208, 308 to assist the call taker 208, 308 in handling the call. The graphical representation includes the mental health codes 118, the user tags 119a, the call taker tags 119b, the call history, as well as a visual summary of the information. Again, since the caller has not requested anonymity, in this instance the mental health history 116 of the caller is also presented to the call taker. The graphical representation may include the mental health history 116 of the caller 120 as well as a visual summary of the information.

At block 606, the content of the call is analyzed (for example via natural language processing or text processing after a voice to text conversion) to identify additional information for including in the mental health history 116 about the mental health of the caller. In some embodiments, the call taker records specific mental health notes about the personal mental health of the caller based on the communication from the caller.

At block 608, as described above, the content of the call is further analyzed (for example via natural language processing or text processing after a voice to text conversion) to identify additional information about the mental health status of the caller. Block 606 and block 608 are executed concurrently.

At block 610 it is determined if the call has progressed (via natural language processing or text processing after a voice to text conversion). For example, the call taker may have asked the caller 120 another question. If the call has progressed, the method returns to block 604 since the caller has not requested anonymity. The mental health codes 118, the user tags 119a, the call taker tags 119b, the call history, and the mental health history 116 presented to the call taker are updated to address the progression of the call.

Once the call has been completed, at block 612 the escrow database 108 is accessed to update the second portion of the mental health profile of the caller 120 with the additional information. If personal mental health information relating to the caller's mental health was collected at block 606, the collected information may be anonymized and locked in the mental health history 116.

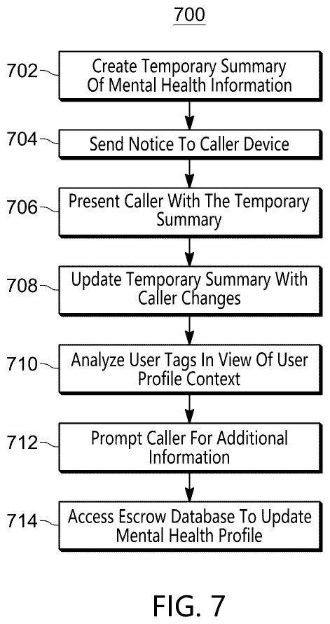

After the user profile at the escrow database 108 has been updated, the caller 120 is provided with an opportunity to review the information stored at the escrow database 108. Referring to FIG. 7, a flowchart representative of a general method for confirming information submitted to the escrow database 108 is illustrated generally by numeral 700. At block 702, a temporary summary of the mental health information for the caller 120 is created. The temporary summary includes any additional personal mental health information collected at block 606 and any additional information collected at block 608. At block 704, an indication that the temporary summary is available is sent to a communication device of the caller 120. The indication includes an actionable link (URL) allowing the caller to review the additional anonymized information. If the collection of personal mental health information was permitted, the caller will also be allowed to review the additional personal mental health information.

When the caller accesses the link, at block 706 the caller is presented with the temporary summary. The caller is provided with an opportunity to add user tags to the user profile 110. The caller is also provided with an opportunity to update the request for anonymity. At block 708, the temporary summary is updated based on any changes or user tags provided by the caller.

At block 710, the added user tags are analyzed in view of the context provided by the user profile 110. For example, consider a user tag “Unable to call from primary phone”. Based on other information in the user profile 110 and the context “primary phone”, a prompt for an alternate phone number is created. Similarly, based on other information in the user profile 110 and the context “unable to call”, a prompt to determine if texting is a viable alternative is created. As another example, consider a user tag “Anxious”. Based on the context “feelings” and on other information in the user profile 110 that “anxious”=“Are you seeking help”, a prompt to enter help number of mental health provider is created. Similarly, based on the context “feelings” and on other information in the user profile 110 that “anxious” =“Do you have people you trust around you?”, a prompt to enter the names of the people and link them to the user profile 110 is created.

At block 712, the prompts are used to collect the required information from the caller in response to the entered user tags. At block 714, once the additional mental health information has been approved by the caller and the user tags have been added, the escrow database 108 is accessed to update mental health profile 110.

Although the specification described that the mental health history 116 stored in the locked portion of the data structure, in some embodiments all, or a portion, of the mental health codes 118, the user tags 119a, the call taker tags 119b, and the call history, can be moved from the accessible portion of the data structure to the locked portion of the data structure. The information can be moved in response to the caller 120 applying greater privacy to the user profile 110. In some embodiments, all, or a portion, of the mental health history 116, the mental health codes 118, the user tags 119a, the call taker tags 119b, and the call history, can be deleted from the user profile 110. In some embodiments, all, or a portion, of the mental health history 116 can be moved from the locked portion of the data structure to the accessible portion of the data structure. The information can be moved in response to the caller 120 applying greater access to the user profile 110.

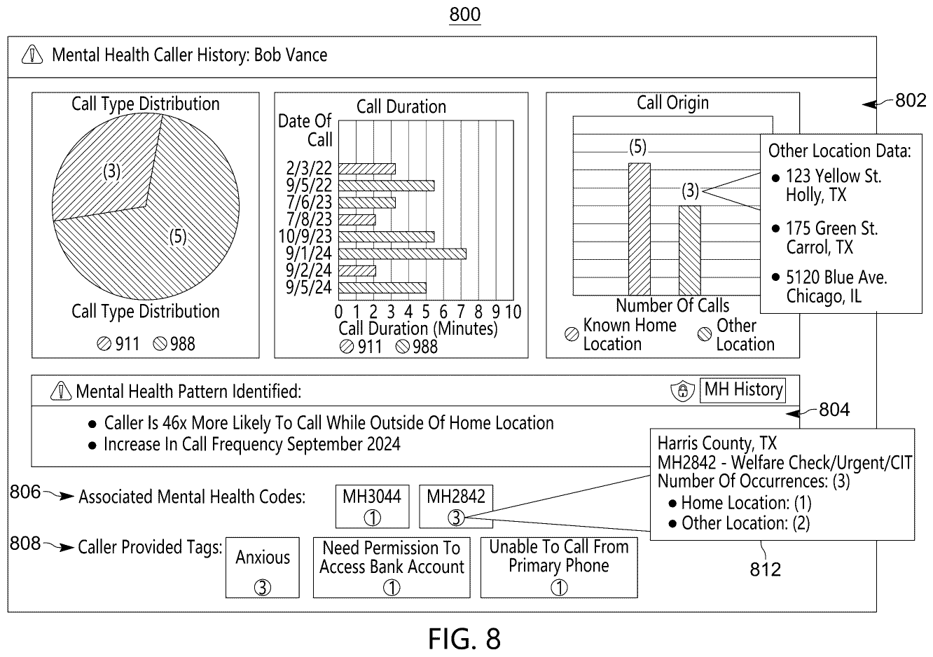

Referring to FIG. 8, an example of the graphical representation of the mental health history 116, the mental health codes 118, the user tags 119a, the call taker tags 119b, the call history is illustrated generally by numeral 800. As described above, the graphical representation 800 includes a visual summary of the mental health information 802, a pattern summary 804 determined from the mental health history 116, a list 806 of the relevant mental health codes, and a list 808 of tags. In this example, the tags are user tags that have been provided by the caller. Selecting a portion of the graphical representation provides further details, if they are available. For example, hovering an input device over one of the mental health codes in the list 806 provides a mental health code detail box 812. The health code detail box 812 identifies the region in which the mental health code was entered, a brief textual description of the mental health code, and historical information about the number and location of occurrences of the mental health code.

As will be appreciated, references in the description to 988 and 911 numbers for crisis prevention centers and emergency dispatch centers are provided for convenience only. Different numbers may be used in different regions. Similarly, reference to 911 are also used for convenience only and can refer to any emergency dispatch system

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. Unless the context of their usage unambiguously indicates otherwise, the articles “a,” “an,” and “the” should not be interpreted as meaning “one” or “only one.” Rather these articles should be interpreted as meaning “at least one” or “one or more.” Likewise, when the terms “the” or “said” are used to refer to a noun previously introduced by the indefinite article “a” or “an,” “the” and “said” mean “at least one” or “one or more” unless the usage unambiguously indicates otherwise.

Also, it should be understood that the illustrated components, unless explicitly described to the contrary, may be combined or divided into separate software, firmware, and/or hardware. For example, instead of being located within and performed by a single electronic processor, logic and processing described herein may be distributed among multiple electronic processors. Similarly, one or more memory modules and communication channels or networks may be used even if embodiments described or illustrated herein have a single such device or element. Also, regardless of how they are combined or divided, hardware and software components may be located on the same computing device or may be distributed among multiple different devices. Accordingly, in this description and in the claims, if an apparatus, method, or system is claimed, for example, as including a controller, control unit, electronic processor, computing device, logic element, module, memory module, communication channel or network, or other element configured in a certain manner, for example, to perform multiple functions, the claim or claim element should be interpreted as meaning one or more of such elements where any one of the one or more elements is configured as claimed, for example, to make any one or more of the recited multiple functions, such that the one or more elements, as a set, perform the multiple functions collectively.

The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “one of”, without a more limiting modifier such as “only one of”, and when applied herein to three or more subsequently defined options such as “one of A, B, and C” should be construed to mean an existence of any one of the options in the list alone (e.g., A alone, B alone, or C alone) or any combination of the three or more of the options in the list (e.g., A and B and C together, A and B together, A and C together, or B and C together).

A device or structure that is “configured” in a certain way is configured in at least that way, but may also be configured in ways that are not listed.

The terms “coupled”, “coupling” or “connected” as used herein can have several different meanings depending on the context in which these terms are used. For example, the terms coupled, coupling, or connected can have a mechanical or electrical connotation. For example, as used herein, the terms coupled, coupling, or connected can indicate that two elements or devices are directly connected to one another or connected to one another through intermediate elements or devices via an electrical element, electrical signal or a mechanical element depending on the particular context.

What is claimed is:

1. A method comprising:
analyzing, at a first emergency services provider, content of communications between a caller experiencing an event and a call taker;
determining an identifier associated with the caller;
accessing an escrow database using the identifier associated with the caller to determine a presence of a behavioural assessment indicator, the behavioural assessment indicator indicating that the caller has a mental health profile stored in the escrow database, wherein the escrow database is generally inaccessible but becomes accessible by predefined, disparate service providers responsive to the caller calling the first emergency services provider;
receiving permission from the caller to access the mental health profile;
retrieving mental health information of the caller from a data structure in the mental health profile, the data structure comprising a first portion storing a locked mental health history of the caller and a second portion storing accessible information including the information about a mental health status of the caller;
presenting, to the call taker, the information about the mental health status of the caller to assist the call taker in handling the call.

2. The method of claim 1, wherein the information from the mental health profile is presented to the call taker dynamically with progression of the communication based on the analysis of the content thereof.

3. The method of claim 1 further comprising:
analyzing the content of communications to identify additional mental health information about the mental health status of the caller; and
accessing the escrow database to update the mental health profile of the caller with the additional mental health information.

4. The method of claim 3 further comprising receiving an indication that the caller has not requested anonymity; and in response to the indication:
analyzing the communication to identify additional information for including in the mental health history; and
accessing the escrow database to update the first portion of the metal health profile of the caller with the additional information.

5. The method of claim 3 further comprising:
presenting the call taker with at least one mental health code based on the analysis of the communication;
provide the call taker with a plurality of tags associated with the at least one mental health code; the tags providing a simplified, readable representation of a mental health condition of the caller.

6. The method of claim 3, further comprising
sending, to a communication device of the caller, an indication that the additional mental health information was identified, the indication including an actionable link allowing the caller to review at least a portion of the additional mental health information; and
storing the additional mental health information in the escrow database upon approval by the caller.

7. The method of claim 1, wherein presenting the mental health profile comprises presenting mental health codes, tags, and historical call data stored in the mental health profile, the tags providing a simplified, readable representation of a mental health condition of the caller.

8. The method of claim 7, wherein the mental health codes are specific to a region from which the mental health codes were originally submitted to the escrow database, the method further comprising:
converting the mental health codes to local mental health codes specific to a region in which the call taker is located; and
presenting the local mental health codes to the call taker.

9. The method of claim 7, further comprising receiving an indication that the caller has not requested anonymity; and in response to the indication:
retrieving the mental health history of the caller from the data structure in the mental health profile; and
presenting to the call taker the mental health history of the caller.

10. The method of claim 7, further comprising presenting an actionable mental health care assist button to the call taker, which when actioned, causes a mental health care professional associated with the stored mental health codes to be conferenced into call.

11. The method of claim 9, further comprising:
dynamically generating a graphical representation of the mental health codes for presenting to the call taker in addition to the mental health codes and the tags associated with the mental health codes; and
if the indication that the caller has not requested anonymity is received, including the mental health history of the caller in dynamically generated graphical representation;
wherein the mental health codes and the tags associated with the mental health codes are displayed so that hovering over them provides an expanded description thereof to the call taker.

12. A device comprising:
a processor; and
a computer readable medium having stored thereon program instructions that, when executed by the processor, cause the processor to perform a set of operations comprising:
analyzing, at a first emergency services provider, content of communications between a caller experiencing an event and a call taker;
determining an identifier associated with the caller;
accessing an escrow database using the identifier associated with the caller to determine a presence of a behavioural assessment indicator, the behavioural assessment indicator indicating that the caller has a mental health profile stored in the escrow database, wherein the escrow database is generally inaccessible but becomes accessible by predefined, disparate service providers responsive to the caller calling the first emergency services provider;
receiving permission from the caller to access the mental health profile;
retrieving mental health information of the caller from a data structure in the mental health profile, the data structure comprising a first portion storing a locked mental health history of the caller and a second portion storing accessible information including the information about a mental health status of the caller;
presenting, to the call taker, the information about the mental health status of the caller to assist the call taker in handling the call.

13. The device of claim 12, wherein the information from the mental health profile is presented to the call taker dynamically with progression of the communication based on the analysis of the content thereof.

14. The device of claim 12, the set of operations further comprising:
analyzing the content of communications to identify additional mental health information about the mental health status of the caller; and
accessing the escrow database to update the mental health profile of the caller with the additional mental health information.

15. The device of claim 14, the set of operations further comprising receiving an indication that the caller has not requested anonymity; and in response to the indication:
analyzing the communication to identify additional information for including in the mental health history; and
accessing the escrow database to update the first portion of the metal health profile of the caller with the additional information.

16. The device of claim 14, the set of operations further comprising:
presenting the call taker with at least one mental health code based on the analysis of the communication;
provide the call taker with a plurality of tags associated with the at least one mental health code; the tags providing a simplified, readable representation of a mental health condition of the caller.

17. The device of claim 13, the set of operations further comprising
sending, to a communication device of the caller, an indication that the additional information was identified, the indication including an actionable link allowing the caller to review at least a portion of the additional mental health information; and
storing the additional mental health information in the escrow database upon approval by the caller.

18. The device of claim 12, wherein presenting the mental health profile comprises presenting mental health codes and tags stored in the mental health database, the tags providing a simplified, readable representation of a mental health condition of the caller.

19. The device of claim 18, wherein the mental health codes are specific to a region from which the mental health codes were originally submitted to the escrow database, the set of operations further comprising:
converting the mental health codes to local mental health codes specific to a region in which the call taker is located; and
presenting the local mental health codes to the call taker.

20. The device of claim 18, the set of operations further comprising receiving an indication that the caller has not requested anonymity; and in response to the indication:
retrieving the mental health history of the caller from the data structure in the mental health profile ; and
presenting to the call taker the mental health history of the caller.

21. The device of claim 18, the set of operation further comprising presenting an actionable mental health care assist button to the call taker, which when actioned, causes a mental health care professional associated with the stored mental health codes to be conferenced into call.

22. The device of claim 20, the set of operations further comprising:
dynamically generating a graphical representation of the mental health codes for presenting to the call taker in addition to the mental health codes and the tags associated with the mental health codes; and
if the indication that the caller has not requested anonymity is received, including the mental health history of the caller in dynamically generated graphical representation;
wherein the mental health codes and the tags associated with the mental health codes are displayed so that hovering over them provides an expanded description thereof to the call taker.
